(19) United States
(12) Patent Application Publication
(10) Pub. No.: US 2004/0248836 A1
(43) Pub. Date: Dec. 9, 2004

(54) Methods for treating diabetes and insulin resistance

(75) Inventor: Cydney C. Brooks, Brighton, MA (US)

Correspondence Address: LAHIVE & COCKFIELD, LLP., 28 STATE STREET, BOSTON, MA, 02109, US

(73) Assignee: AdipoGenix, INC., Boston, MA

(21) Appl. No.: 10/627,310

(22) Filed: Jul. 25, 2003

Related U.S. Application Data
(60) Provisional application No. 60/401,389, filed on Aug. 5, 2002.

(51) Int. Cl. A61K 48/00; C12Q 1/68

(52) U.S. Cl. 514/44; 435/6

(57) ABSTRACT

Methods for treating diabetes and/or insulin resistance by administering an activator of FHOS are disclosed.

### REFERENCE TO RELATED APPLICATIONS

The present invention claims priority to a U.S. Provisional Patent Application, Ser. No. 60/401,389, filed Aug. 5, 2002, entitled Methods for Treating Diabetes and Insulin Resistance. Provisional Patent Application Ser. No. 60/401,389 is hereby incorporated by reference.

### BACKGROUND OF THE INVENTION

Approximately 17 million persons in the United States are affected by type II diabetes, also known as adult-onset or non-insulin dependent diabetes mellitus (NIDDM). Type II diabetes is a chronic and incurable disease that accounts for 90% to 95% of all diagnosed cases of diabetes. It is a leading cause of death in the United States. This condition is a complex disorder involving a combination of factors, including the inability of certain tissues to respond to insulin, referred to as “insulin resistance” and an inability of the pancreas to produce appropriate levels of insulin. Insulin resistance occurs when insulin-stimulated uptake of glucose into peripheral cells is impaired and the stimulatory effect of insulin on glucose production in the liver is reduced. This condition can lead to excessive blood levels of glucose (hyperglycemia). Hyperglycemia in most diabetics is accompanied by, and may even be preceded by, increases in the blood levels of triglycerides, free fatty acids, and other lipids. Hyperglycemia over an extended period of time is believed to damage the walls of blood vessels, causing complications characteristic of diabetes, including blindness from microvascular deterioration in the retina, loss of circulation in the extremities leading to amputation, and kidney failure. In addition, the high blood levels of triglycerides, free fatty acids, and total cholesterol pose serious health risks to the diabetic and are believed to lead to cardiovascular disease including coronary heart disease.

Environmental factors, especially diet, physical activity, and age are known to affect the timing of onset as well the severity of type II diabetes. Moreover, it is believed that both insulin resistance and type II diabetes are, at least in part, genetically regulated. In spite of intense investigation, however, the genes responsible for the development of type II diabetes remain unknown.

Thus there exists a need for the identification of genes and/or proteins involved in the development of both insulin resistance and type II diabetes. New and innovative treatments for insulin resistance and/or diabetes are clearly a priority for researchers in this field.

### SUMMARY OF THE INVENTION

The present invention provides a method of treating diabetes and related diseases or disorders, such as insulin resistance, by administering to a subject an activator of FHOS (Formin Homologue Overexpressed in Spleen). Suitable activators of FHOS which can be employed in the methods of the invention include, but are not limited to, a FHOS nucleic acid molecule, a plasmid comprising a FHOS nucleic acid molecule, a FHOS adenovirus, a FHOS retrovirus and a FHOS protein or biologically active portion thereof. Suitable activators of FHOS further include, but are not limited to a protein or biologically active fragment thereof, a nucleic acid molecule or biologically active fragment thereof, an antibody or biologically active fragment thereof, a peptide, a peptidomimetic, a non-peptide oligomer or small molecule.

Such methods can be used, not only to treat diabetes and related diseases, but also to treat several systemic problems resulting from diabetes, such as hyperglycemia.

In another aspect, the invention provides method of treating diabetes by administering to a subject an activator of FHOS, either alone or in combination with other diabetes therapeutics.

Screening assays for identifying compounds suitable for treatment of diabetes and/or insulin resistance are also provided.

### BRIEF DESCRIPTION OF THE DRAWINGS

FIG. 1 is a graph showing the correlation between decreased FHOS mRNA levels in adipocytes from diabetic and insulin resistant subjects as compared to control, lean subjects.

FIG. 2 is a graph depicting the data of FIG. 1 in an averaged format.

FIG. 3 depicts the nucleotide and amino acid sequence of human FHOS. FIG. 3A-B depicts the nucleotide sequence and FIG. 3C depicts the amino acid sequence.

### DETAILED DESCRIPTION OF THE INVENTION

FHOS (Formin Homologue Overexpressed in Spleen), originally identified by Westendorf et al., (1 999) Gene 232:173182) is a formin/diaphanous protein family member. Diaphanous-related formins are involved in cytokinesis and stress fiber formation and have been shown to bridge Rho GTPase and Src tyrosine kinase signaling (Tominaga et al., (2000) Mol. Cell 5:13-25). FHOS specifically has been shown to interact with Rac 1 and activate transcription from the serum response elements (SRE) (Westendorf, (2001) J Biol Chem 276:46453-46459).

The present invention is based, in part, on the surprising discovery that FHOS expression is downregulated in the adipocytes of human subjects having diabetes and/or insulin resistance. Thus the present invention is directed to methods of increasing expression of FHOS, for example, in fat and/or muscle cells of diabetic and/or insulin resistant subjects as a therapeutic approach to treating said subjects.

Accordingly, in one embodiment, the present invention provides a method for treating type II diabetes by administering to a subject an activator of FHOS in an amount sufficient to restore normal levels of FHOS in said subject. Preferably, an FHOS activator is administrated in an amount sufficient to ameliorate at least one symptom of the disease. Related disorders, such as obesity or hyperglycemia can also be treated according to the present invention by administration of a FHOS activator.

FHOS activators for use in the methods of the present invention can be identified using a variety of appropriate bioassays which test for the ability to stimulate FHOS expression or biological activity. The ability of the FHOS activators to stimulate FHOS expression or activity is preferably specific, i.e., the FHOS activator can specifically stimulate FHOS mRNA or protein expression or FHOS protein activity.

So that the invention may be more readily understood, certain terms are first defined.

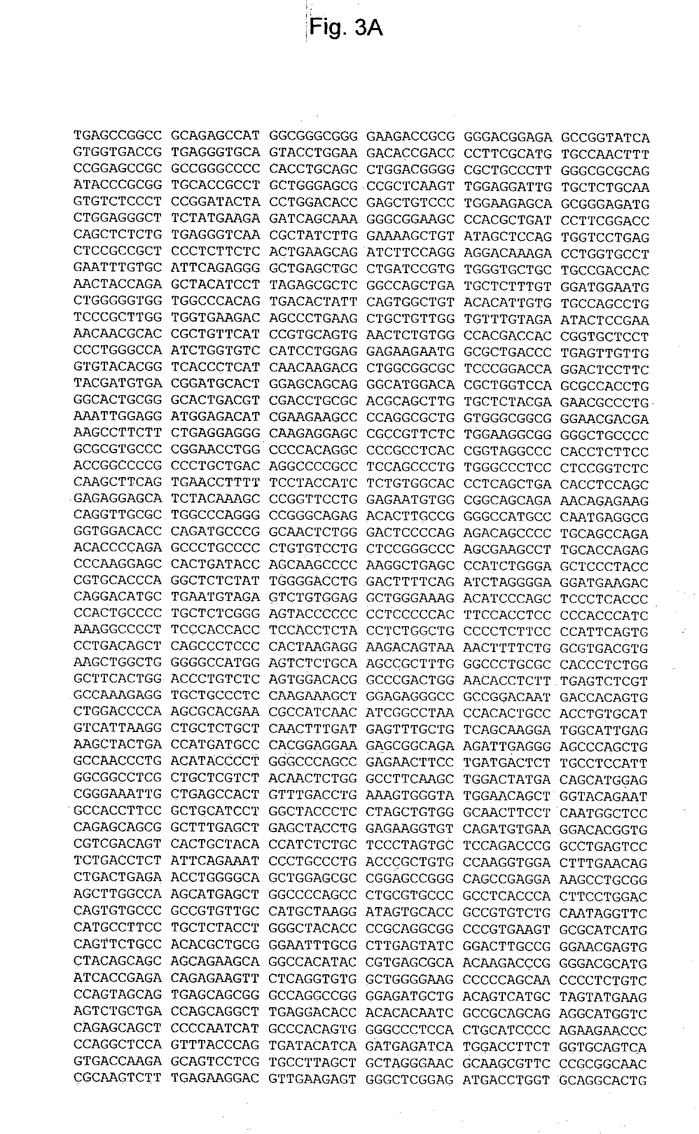

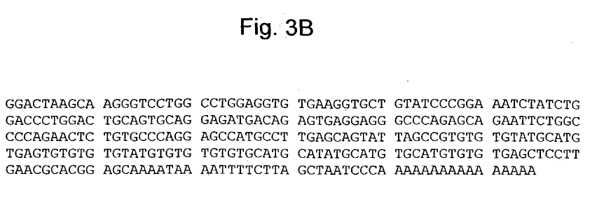

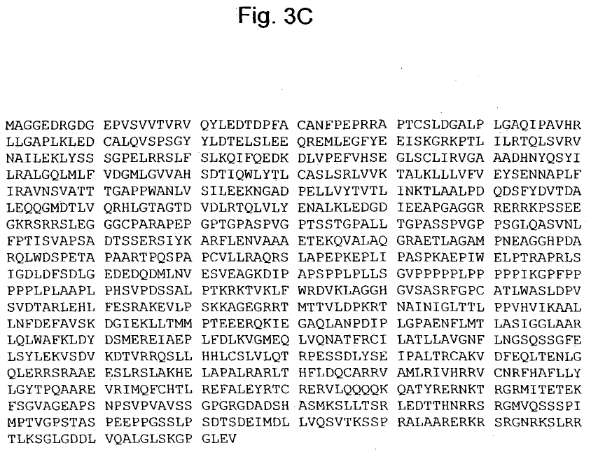

As used herein, the term “FHOS” includes all known forms of FHOS and preferably refers to human FHOS. The term “FHOS protein” refers to any form of FHOS polypeptide, for example, full-length FHOS polypeptides and FHOS fragments (e.g., bioactive fragments, structural and/or functional domains, and the like). The term “FHOS nucleic acid molecule” refers to any form of FHOS polynucleotide, for example, polynucleotides encoding full-length FHOS proteins, polynucleotides encoding FHOS fragments (e.g., bioactive fragments, structural and/or functional domains, and the like), and FHOS non-coding polynucleotides (e.g., 5′ untranslated regions or 3′ untranslated regions, regulatory sequences, and the like). The human nucleic acid and amino acid sequences for FHOS are shown in FIG. 3A-B and 3C, respectively.

As used herein, the term “FHOS activator” or “activator of FHOS” includes any agent capable of activating FHOS mRNA and/or protein expression, including but not limited to peptides, peptidomimetics, antibodies or fragments thereof, oligonucleotides, or other small molecules which specifically increase FHOS mRNA and/or protein levels in the cells (e.g., fat and/or muscle cells) of a subject, e.g., a human subject. The term “FHOS activator” also encompasses FHOS nucleic acid molecules (or fragments of said nucleic acid molecules) including nucleic acid molecules in vectors, as described herein, and FHOS protein or polypeptides themselves (or fragments of said proteins or polypeptides) as agents suitable for increasing FHOS levels in the cells (e.g., fat and/or muscle cells) of a subject. The term “FHOS activator” or “activator of FHOS” also includes any agent capable of activating FHOS activity, including but not limited to proteins (or fragments thereof), peptides, peptidomimetics, antibodies or fragments thereof, nucleic acid molecules (or fragments thereof), oligonucleotides, or other small molecules which specifically increase or enhance the action of FHOS.

As used herein, the term “FHOS activity” includes any biological or physiological activity mediated by FHOS. The activities of FHOS are described, for example, in Westendorf et al. (1999) Gene 232:173-182, Tominaga et al. (2000) Mol. Cell 5:13-25 and Westendorf (2001) J. Biol. Chem. 276:46453-46459, the entire contents of which are incorporated herein by this reference. Activity can be assessed by any art-recognized methodology.

As used herein, the term “bioassay” includes any assay designed to identify a FHOS activator. The assay can be an in vitro or an in vivo assay suitable for identifying whether a FHOS activator can stimulate FHOS expression and/or stimulate one or more of the biological functions of FHOS.

As used herein, the term “modulate” means to increase (or activate or upregulate) or decrease (or inhibit or downregulate). For example, modulation of gene expression refers to an activation or upregulation or inhibition or downregulation of expression. Modulation of gene expression includes both direct and indirect modulation of expression. For example, modulation of gene expression includes, but is not limited to, modulation of RNA transcription, modulation of regulatory factors involved in FHOS transcription (e.g., activators or co-activators), modulation of regulatory factors involved in RNA degradation, and the like. Modulation of protein expression refers to an activation or upregulation or inhibition or downregulation of protein expression. Modulation of protein activity refers to an activation or upregulation or inhibition or downregulation of protein activity. Modulation of gene expression or protein expression or activity is readily detectable as a change in, for example, FHOS mRNA and/or protein levels. Modulation of, for example, protein expression, may also be detectable as a change in processing or cellular localization of a protein. As used herein, the term “activation” refers to any upregulation, whether partial or whole, resulting in increased mRNA and/or protein levels.

As used herein, the term “diabetes” includes all known forms of diabetes, including type I and type II diabetes, as described in Abel et al., Diabetes Mellitus: A Fundamental and Clinical Text (1996) pp.530-543.

FHOS activators of the invention are typically administered to a subject in “substantially pure” form. The term “substantially pure” as used herein refers to an FHOS activator which is substantially free of other molecules or materials with which it is naturally associated. One skilled in the art can purify FHOS activators using standard purification techniques. For example, when the FHOS activator is a gene therapy vector, the substantially pure polynucleotide will yield a single major band on, an agarose gel. The purity of an FHOS gene therapy vector can also be determined by restriction mapping. When the FHOS activator is a FHOS protein or polypeptide, the substantially pure protein or polypeptide will yield a single major band on a non-reducing polyacrylamide gel. The purity of a FHOS protein or polypeptide can also be determined by amino-terminal amino acid sequence analysis.

As used herein, the term “modulation of FHOS activity” or “modulation of FHOS level” refers to a change in FHOS activity or level compared to its native state. This change may be either positive (upregulation), or negative (downregulation), but for the purposes of the present invention is preferably the former.

Cells which are targeted by the methods of the present invention, such as muscle and fat cells, include isolated cells maintained in culture as well as cells within their natural context in vivo (e.g., in fat tissue or muscle tissue, such as pectoralis, triceps, gastrocnemius, quadriceps, and iliocostal muscles).

Gene Therapy

In one aspect of the invention, a gene construct is used as a part of a gene therapy protocol to deliver a nucleic acid encoding a FHOS protein, or a biologically active portion thereof. Accordingly, the invention features expression vectors for in vivo or in vitro transfection and expression of FHOS (or a biologically active portion thereof) in particular cell types so as to increase the activity of FHOS in said cell. Such therapies are particularly useful where the naturally-occurring form of the protein is misexpressed or inappropriately activated.

Expression constructs encoding FHOS (or a biologically active portion thereof) may be administered in any biologically effective carrier, e.g. any formulation or composition capable of effectively delivering the recombinant gene to cells in vivo. Approaches include insertion of the DNA encoding FHOS (or a biologically active portion thereof) in viral vectors including recombinant retroviruses, adenovirus, adeno-associated virus, and herpes simplex virus-1, or recombinant bacterial or eukaryotic plasmids. Vital vectors infect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or Ca₂PO4 precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically. Furthermore, it will be recognized that particular gene constructs provided for in vivo transduction of FHOS expression are also useful for in vitro transduction of cells, such as for use in the ex vivo tissue culture systems described below.

A preferred approach for in vivo introduction of nucleic acid into a cell is by use of a viral vector containing nucleic acid, e.g. a cDNA, encoding the FHOS protein (or a biologically active portion thereof). Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for,a review see Miller (1990) Blood 76:271). Thus, recombinant retrovirus can be constructed in which part of the retroviral coding sequence (gag, pol, env) has been replaced by nucleic acid encoding one of the subject proteins rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψ Crip, ψCre, ψ2 and ψAm. Retroviruses have been used to introduce a variety of genes into many different cell types, including neuronal cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) PNAS 86:9079-9083; Julan et al. (1992) J. Gen Virol 73:3251-3255; and Goud et al. (1983) Virology 163:251-254); or coupling cell surface receptor ligands to the viral env proteins (Neda et al. (1991) J Biol Chem 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector into an amphotropic vector.

Moreover, use of retroviral gene delivery can be further enhanced by the use of tissue- or cell-specific transcriptional regulatory sequences that control expression of the FHOS gene of the retroviral vector.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) Biotechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses can be advantageous in certain circumstances in that they can be used to infect a wide variety of cell types, including airway epithelium (Rosenfeld et al. (1992) cited supra), endothelial cells (Lemarchand et al. (1 992) Proc. Natl. Acad. Sci. USA 89:6482-6486), adipocytes (Hertzel et al. (2000) J. Lipid Res. 41:1082-1086), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the inserted FHOS gene can be under control of, for example, the E1A promoter, the major late promoter (MLP) and associated leader sequences, the E3 promoter, or exogenously added promoter sequences.

Yet another viral vector system useful for delivery of the DNA encoding FHOS (or a biologically active portion thereof) is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of a FHOS (or a biologically active portion thereof) in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject FHOS-encoding DNA by the targeted cell. Exemplary gene delivery systems of this type include liposomal derived systems, poly-lysine conjugates, and artificial viral envelopes.

In clinical settings, the gene delivery systems for therapeutic FHOS administration can be introduced into a patient by any of a number of methods, each of which is familiar in the art. For instance, a pharmaceutical preparation of the gene delivery system can be introduced systemically, e.g. by intravenous injection. In this case, specific transduction of the protein in the target cells arises from specificity of transfection provided by the gene delivery vehicle, (i.e., cell-type or tissue-type specificity due to transcriptional regulatory sequences controlling recombinant gene expression). In other embodiments, initial delivery of the recombinant gene is more limited with introduction into the animal being quite localized. For example, the gene delivery vehicle can be introduced by catheter (see U.S. Pat. 5,328,470) or by stereotactic injection (e.g. Chen et al. (1994) PNAS 91: 3054-3057). The DNA encoding FHOS (or a biologically active portion thereof), can be delivered in a gene therapy construct by electroporation using techniques described, for example, by Dev et al. ((1994) Cancer Treat Rev 20:105-115).

The present invention also includes therapeutic methods comprising injecting an area of a subject's body with an effective amount of a naked plasmid DNA compound (such as is taught, for example in Wolff et al., (1990) Science 247:1465-1468). A naked plasmid DNA compound comprises a nucleic acid molecule encoding a FHOS protein or biologically active portion thereof, operatively linked to a naked plasmid DNA vector capable of being taken up by and expressed in a recipient cell located in the body area. Preferred naked plasmid DNA vectors of the present invention include those known in the art. When administered to a subject, a naked plasmid DNA compound of the present invention transforms cells within the subject and directs the production of FHOS protein, or biologically active portion thereof, in the cell.

A naked plasmid DNA compound of the present invention can be injected directly into fat and/or muscle cells or a subject in an amount such that the plasmid is taken up and expressed by the fat and/or muscle cells. As used herein, an effective amount of a naked plasmid DNA to administer to a subject comprises an amount needed to alleviate at least one symptom of the disease or disorder being treated and, preferably, is an amount sufficient to prevent or cure the disease or disorder. The mode of administration, number of doses and frequency of dose capable of being decided upon, in any given situation, by one of skill in the art without resorting to undue experimentation.

The present invention also includes therapeutic methods comprising administering to a subject a genetically-engineered human cell, for example, a genetically-engineered muscle cell or adipocyte, wherein the cell is engineered to overexpress the FHOS gene. The terms “genetically-engineered cell” and “recombinant cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

In one embodiment, a recombinant expression vector is introduced into the cell, the vector containing a nucleic acid molecule which encodes a FHOS protein (or a biologically active portion thereof). As used herein, the term “vector” refers to a nucleic acid molecule capable of transporting another nucleic acid to which it has been linked. A preferred vector is an “expression vector” which is capable of directing the expression of gene contained therein. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. In a preferred embodiment, genetic engineering is of a subject or patient's own cells which are isolated from the subject or patient's body, transfected or infected according to the techniques described in detail herein, and reintroduced or returned to the body of the subject or patient.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology. Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like.

When using in mammalian cells, e.g., human cells, the expression vector's control functions are often provided by viral regulatory elements. For example, commonly used promoters are derived from polyoma, Adenovirus 2, cytomegalovirus and Simian Virus 40. Alternatively, tissue-specific regulatory elements are used to control expression of the FHOS-encoding nucleic acid. Tissue-specific regulatory elements are known in the art. Preferred tissue-specific promoters include fat-specific promoters and muscle-specific promoters.

Vector DNA can be introduced via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning. A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding the FHOS protein or can be introduced on a separate vector. Cells stably transfected with the introduced nucleic acid can be identified by drug selection (e.g., cells that have incorporated the selectable marker gene will survive, while the other cells die).

Alternatively, the expression of an endogenous FHOS gene can be modified, e.g., increased, within a cell by inserting a heterologous DNA regulatory element into the genome of the cell such that the inserted regulatory element is operatively linked with the endogenous FHOS gene. For example, an endogenous FHOS gene may be activated by inserting a regulatory element that is capable of promoting the expression of a normally expressed gene product in the cell. The heterologous regulatory element is inserted using techniques, such as targeted homologous recombination, which are well known to those of skill in the art, and described, e.g., in Chappel, U.S. Pat. No. 5,272,071; PCT publication No. WO 91/06667, published May 16, 1991.

Genetically-engineered cells are administered to a subject in need thereof, e.g., a subject having diabetes or an insulin-resistant subject, utilizing any art-recognized method for administering cells to a patient (see e.g., U.S. Pat. No. 5,538,722). In a preferred embodiment, cells are administered via injection, for example, via injection into fat or muscle tissue of the subject in need of treatment.

Protein Therapy

The present invention also includes therapeutic methods comprising administering to a subject a therapeutically effective dose of FHOS protein or a biologically active portion thereof, such that FHOS protein levels in said subject are increased or restored to levels detectable in normal or control (e.g., lean) individuals. Preferably, the FHOS protein or biologically active portion thereof is made via recombinant means. Biologically active fragments (or portions) of FHOS are produced by expression of a fragment (or portion) of a FHOS-encoding nucleic acid molecule such that the FHOS protein fragment (or portion) is produced recombinantly. Biologically active fragments (or portions) of FHOS can be produced by digestion of native or recombinantly produced FHOS by, for example, using a protease, e.g., trypsin, thermolysin, chymotrypsin, or pepsin. Computer analysis (using commercially available software, e.g. MacVector, Omega, PCGene, Molecular Simulation, Inc.) can be used to identify proteolytic cleavage sites.

Recombinant proteins (or fragments) can be made according to any well-established methodology for expressing and purifying such proteins. For example, recombinant expression vectors can be designed for expression of FHOS protein in prokaryotic or eukaryotic cells. For example, FHOS protein can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Recombinant expression vectors for expression of FHOS protein in eukaryotic cells are described below. More routinely, however, recombinant FHOS proteins are produced in prokaryotic cells, for example E. coli cells. Examples of suitable E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA modification and synthesis techniques, e.g., mutagenesis techniques.

In another embodiment, the FHOS expression vector is a yeast expression vector. Examples of vectors for expression in yeast S. cerevisiae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, FHOS protein can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

A prokaryotic, yeast or insect cell (into which a recombinant expression vector encoding a FHOS protein has been introduced) is then cultured in a suitable medium such that the FHOS protein is produced and the FHOS protein is then isolated or purified from the medium or the host cell. When the FHOS protein or biologically active portion thereof is recombinantly produced, it is also preferably substantially free of culture medium, i.e., culture medium represents less than about 20%, more preferably less than about 10%, and most preferably less than about 5% of the volume of the protein preparation. In some instances it may be desirable to utilize a solubilizing agent such that the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

The invention also provides for reduction of the FHOS proteins to generate mimetics, e.g. peptide or non-peptide agents, which are able to function in a manner similar to naturally-occurring FHOS yet have improved therapeutic properties as compared to naturally-occurring FHOS. A mimetic can be obtained by, for example, screening libraries of natural and synthetic compounds as disclosed herein that are capable of functioning in a manner similar to naturally-occurring FHOS. A mimetic can also be obtained by, for example, rational drug design.

FHOS Activating Antibodies

The present invention also includes therapeutic methods comprising administering to a subject a therapeutically effective dose of an FHOS activating antibody or biologically active portion thereof, such that FHOS biological activity in said subject is increased or restored to levels detectable in normal or control (e.g., lean) individuals. Preferred antibodies include monoclonal antibodies, including humanized, chimeric and human monoclonals or fragments thereof. To generate such antibodies, a proteolytic or synthetic FHOS fragment (alone or linked to a suitable carrier or hapten) can be used to immunize a subject (e.g., a mammal including, but not limited to a rabbit, goat, mouse or other mammal). For example, the methods described in U.S. Pat. Nos. 5,422,110; 5,837,268; 5,708,155; 5,723,129; and 5,849,531, can be used and are incorporated herein by reference. The immunogenic preparation can further include an adjuvant, such as Freund's complete or incomplete adjuvant, or similar immunostimulatory agent. Immunization of a suitable subject with an immunogenic proteolytic or synthetic FHOS fragment preparation induces a polyclonal anti-FHOS antibody response. The anti-FHOS antibody titer in the immunized subject can be monitored over time by standard techniques, such as with an enzyme linked immunosorbent assay (ELISA) using immobilized FHOS. Subsequently, the sera from the immunized subjects can be tested for their FHOS stimulatory activity using any of the bioassays described herein.

Alternatively, it is also possible to immunize subjects with plasmids expressing FHOS using DNA immunization technology, such as that disclosed in U.S. Pat. No. 5,795,872, Ricigliano et al., “DNA construct for immunization” (1998), and in U.S. Pat. No. 5,643,578, Robinson et al., “Immunization by inoculation of DNA transcription unit” (1997).

The antibody molecules directed against FHOS can be isolated from the mammal (e.g., from the blood) and further purified by well known techniques, such as protein A chromatography to obtain the IgG fraction. At an appropriate time after immunization, e.g., when the anti-FHOS antibody titers are highest, antibody-producing cells can be obtained from the subject and used to prepare e.g., monoclonal antibodies by standard techniques, such as the hybridoma technique originally described by Kohler and Milstein (1975) Nature 256:495-497) (see also, Brown et al. (198 1) J. Immunol. 127:539-46; Brown et al. (1980) J. Biol. Chem 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hyhridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies. A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a FHOS immunogen as described above, and the culture supernatants of the resulting hybridoma cells are screened to identify a hybridoma producing a monoclonal antibody that binds FHOS.

Any of the many well known protocols used for fusing lymphocytes and immortalized cell lines can be applied for the purpose of generating an anti-FHOS monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind FHOS, e.g., using a standard ELISA assay. The antibodies can then be tested for FHOS stimulatory activity using, for example, the assays described herein.

Methods for Identifying Additional FHOS Modulators

The invention provides a method (also referred to herein as a “screening assay”) for identifying modulators, i.e., candidate or test compounds or agents (e.g., proteins or protein fragments, peptides, peptidomimetics, nucleic acid molecules, nucleic acid fragments, antibodies or antibody fragments, small molecules or other drugs) that bind to FHOS proteins or have a stimulatory effect on, for example, FHOS expression or FHOS activity.

Test compounds can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). The term “small molecule”, as used herein, includes any biological or chemical molecule having a molecular weight of less than 500 Dalton.

Examples of methods for the synthesis of molecular libraries can be found in the art, for example in: DeWitt et al. (1993) Proc. Natl. Acad. Sci. U S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage ((Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)). In a preferred embodiment, the library is a natural product library, e.g., a library produced by a bacterial, fungal, or yeast culture. In another preferred embodiment, the library is a synthetic compound library.

In one embodiment, an assay is a cell-based assay in which a cell which expresses FHOS is contacted with a test compound and the ability of the test compound to modulate, e.g., increase, FHOS expression and/or activity determined. Determining the ability of the compound to modulate, e.g., increase, FHOS expression can be accomplished, for example, by detecting the presence or absence or amount of a FHOS transcript or protein (e.g., using a probe based on the nucleotide sequences of the present invention or an anti-FHOS antibody). Alternatively, the ability of the compound to modulate expression can be determined using a standard transcription-based assay or reporter gene assay. Reporter gene constructs are prepared by operatively linking a reporter gene with at least one transcriptional regulatory element, e.g., a FHOS transcriptional regulatory element. Transcriptional control elements include, but are not limited to, promoters, enhancers, and repressor and activator binding sites. Reporter genes include any gene that expresses a detectable gene product, which may be RNA or protein. Preferred reporter genes are those that are readily detectable. The reporter gene may also be included in the construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979) Nature 282: 864-869) luciferase, and other enzyme detection systems, such as beta-galactosidase; firefly luciferase (deWet et al. (1987) Mol. Cell Biol. 7:725-737); bacterial luciferase (Engebrecht and Silverman (1984) Proc. Natl. Acad. Sci. USA 1:4154-4158; Baldwin et al. (1984) Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368) and green fluorescent protein (U.S. Pat. No. 5,491,084; WO96/23898).

Modulation of FHOS expression can be either direct or indirect, for example, via modulation of transrciption or translation or by modulation of mRNA and/or protein degradation. In one embodiment, the level of expression of FHOS mRNA or protein in the presence of the candidate compound is compared to the level of expression of FHOS mRNA or protein in the absence of the candidate compound. The candidate compound can then be identified as a modulator of FHOS expression based on this comparison. For example, when expression of FHOS mRNA or protein is greater (statistically significantly greater) in the presence of the candidate compound than in its absence, the candidate compound is identified as a stimulator of FHOS mRNA or protein expression. Alternatively, when expression of FHOS mRNA or protein is less (statistically significantly less) in the presence of the candidate compound than in its absence, the candidate compound is identified as an inhibitor of FHOS mRNA or protein expression. The level of FHOS mRNA or protein expression in the cells can be determined by any art-recognized method for detecting mRNA or protein. For example, mRNA levels can be determined by Northern blot analysis, dot blot analysis, PCR analysis and the like. FHOS protein levels can be determined, for example, by Western blot analysis, immunoprecipitation, peptide mapping and/or sequencing, and the like. mRNA expression can also be determined using reporter gene assays, also referred to herein as transcription-based assays. Preferred reporter gene assay systems include, but are not limited to the chloramphenicol acetyltransferase (CAT) and luciferase enzyme assays systems. Preferred cells include, but are not limited to, mammalian and/or yeast cells.

In another embodiment, an assay is a cell-based assay comprising contacting a cell expressing FHOS and an FHOS target molecule with a test compound and determining the ability of the test compound to modulate (e.g., stimulate) the activity of the target molecule. Determining the ability of the test compound to modulate the activity of the target molecule can be accomplished, for example, by determining the ability of FHOS to bind to or interact with the target molecule or by determining the activity of the target molecule.

In another embodiment, the invention provides assays for screening candidate or test compounds which bind to or increase the activity of a FHOS protein or biologically active portion thereof. Determining the ability of the test compound to bind to the FHOS protein (or biologically active portion thereof) can be accomplished, for example, by coupling the test compound with a radioisotope or enzymatic label such that binding of the test compound to the FHOS protein (or biologically active portion thereof) can be determined by detecting the labeled compound in a complex. For example, test compounds can be labeled with 125I, 35S, 14C, or 3H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test compound to interact with the FHOS protein (or biologically active portion thereof) without the labeling of any of the interactants.

In yet another embodiment, an assay of the present invention is a cell-free assay in which the FHOS protein or biologically active portion thereof is contacted with a test compound and the ability of the test compound to bind to the FHOS protein or biologically active portion thereof is determined. Binding of the test compound to the FHOS protein can be determined either directly or indirectly as described above. In a preferred embodiment, the assay includes contacting the FHOS protein or biologically active portion thereof with a known compound which binds FHOS to form an assay mixture, contacting the assay mixture with a test compound, and determining the ability of the test compound to interact with the FHOS protein, wherein determining the ability of the test compound to interact with the FHOS protein comprises determining the ability of the test compound to preferentially bind to FHOS or biologically active portion thereof as compared to the known compound. The known compound can be, for example, a protein or protein fragment, peptide, peptidomimetic, nucleic acid molecule, nucleic acid fragment, antibody or antibody fragment, small molecule or other agent, demonstrated to bind FHOS either in vitro or in vivo.

In another embodiment, the assay is a cell-free assay in which the FHOS protein or biologically active portion thereof is contacted with a test compound and the ability of the test compound to modulate (e.g., stimulate) the activity of the FHOS protein or biologically active portion thereof is determined. Determining the ability of the compound to modulate, e.g., increase, FHOS activity can be accomplished, for example, by detecting FHOS activity in cellular supernatants (e.g., contacting a second cell with the supernatants). Determining the ability of the test compound to modulate (e.g., stimulate) the activity of FHOS can also be accomplished using any art-recognized assay for determining FHOS activity. The activities of FHOS are described, for example, in Westendorf et al. (1999) Gene 232:173-182, Tominaga et al. (2000) Mol. Cell 5:13-25 and Westendorf (2001) J. Biol. Chem. 276:46453-46459, the entire contents of which are incorporated herein by this reference.

In yet another embodiment, the assay is a cell-free assay in which a composition comprising FHOS and an FHOS target molecule is contacted with a test compound and the ability of the test compound to affect binding of FHOS to the target molecule or to affect target molecule activity is determined.

In more than one embodiment of the above assay methods of the present invention, it may be desirable to immobilize either FHOS or its target molecule to facilitate separation of complexed from uncomplexed forms of one or both of the proteins, as well as to accommodate automation of the assay. Binding of a test compound to FHOS, or interaction of FHOS with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/ FHOS fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or FHOS protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of FHOS binding or activity determined using standard techniques.

Other techniques for immobilizing proteins on matrices can also be used in the screening assays of the invention. For example, either FHOS or its target molecule can be immobilized utilizing conjugation of biotin and streptavidin. Biotinylated FHOS or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with FHOS or target molecules but which do not interfere with binding of the FHOS protein to its target molecule can be derivatized to the wells of the plate, and unbound target or FHOS trapped in the wells by antibody conjugation. Methods for detecting such complexes, in addition to those described above for the GST-immobilized complexes, include immunodetection of complexes using antibodies reactive with the FHOS or target molecule, as well as enzyme-linked assays which rely on detecting an enzymatic activity associated with the FHOS or target molecule.

This invention further pertains to novel agents identified by the above-described screening assays. Accordingly, it is within the scope of this invention to further use an agent identified as described herein in an appropriate animal model. For example, an agent identified as described herein (e.g., a FHOS activator) can be used in an animal model to determine the efficacy, toxicity, or side effects of treatment with such an agent. Alternatively, an agent identified as described herein can be used in an animal model to determine the mechanism of action of such an agent. Preferred animal models include but are not limited to ob/ob, db/db, and mice carrying the lethal yellow mutation. Additional preferred animal models include but are not limited to streptozotocin-induced diabetic rats, obese Zucker rats, and insulin-resistant Otsuka Long-Evans Tokushima Fatty (OLETF) rats.

Functional assays for diabetes include, but are not limited to, an insulin sensitivity assay, a glucose tolerance assay, and an ex-vivo glucose uptake by isolated muscle assay also can be performed to monitor the effect of the agent on treated and non-treated animals. Similarly, obesity can be assayed for in these animals, for example, by measuring serum levels of known molecular markers of obesity, such as glucose, insulin, lipids, triglycerides and creatine kinase. Moreover, body weight, and/or fat pad weights can be assayed in these animals. Alternatively, muscle and fat cell differentiation can be observed in these animals. Analysis of such studies should enable a determination of the overall effect of the activator on the disease phenotype in these animal models. Furthermore, this invention pertains to uses of novel agents identified by the above-described screening assays for treatments as described herein.

Therapeutic Methods

The present invention features methods of treatment or therapeutic methods. In one embodiment, the invention features a method of treating a subject (e.g., a human subject in need thereof) with a FHOS activator such that a desired therapeutic effect is achieved. In another embodiment, the method involves administering to a tissue or cell line, e.g., a tissue or cell line an isolated from a subject or patient, a FHOS activator such that a desired effect (e.g., FHOS mRNA or protein activation) is achieved. In another embodiment, the method involves genetically-engineering a tissue or cell line, e.g., a tissue or cell line from a subject or patient, such that FHOS expression or activity is activated. Tissue or cell lines treated ex vivo with a FHOS activator or genetically-engineered in accordance with the methodologies of the present invention are preferably introduced into the subject or patient after ex vivo manipulation, such that a desired therapeutic effect is achieved.

In a preferred embodiment, the invention features a method of treating a subject having an insulin response disorder, for example, reduced insulin sensitivity or insulin resistance or diabetes (e.g., Type II diabetes). The present invention also provides for therapeutic methods of treating a subject having pre-diabetes or symptoms thereof, hyperglycemia and/or Type I diabetes. Desired therapeutic effects include an increase in FHOS mRNA expression, FHOS protein levels, or FHOS activity, as described herein. Desired therapeutic effects also include, but are not limited to curing or healing the subject, alleviating, relieving, altering or ameliorating a disease or disorder in the subject or at least one symptom of said disease or disorder in the subject, or otherwise improving or affecting the health of the subject.

Identification or selection of a subject in need thereof can be accomplished by any skilled medical practitioner or researcher using art-recognized diagnostic skills or techniques. A diabetic subject is a subject, e.g., a human subject, who has been diagnosed as having diabetes (or would be diagnosed as having diabetes) by a skilled medical practitioner or researcher. Preferred tests utilized in diabetes diagnosis include the fasting plasma glucose (FPG)test and the glucose tolerance test, e.g., the 75-g oral glucose tolerance test (OGTT). Exemplary criteria for the diagnosis of diabetes are set forth below.

| Normoglycemia | IFG or IGT^{‡} | Diabetes* |
|---|---|---|
| FPG < 110 mg/dl | FPG ≧ 110 and | FPG ≧ 126 mg/dl |
| | <126 mg/dl (IFG) | |
| 2-h PG^{†} < 140 mg/dl | 2-h PG^{†} ≧ 140 and | 2-h PG^{†} ≧ 200 mg/dl |
| | <200 mg/dl (IGT) | |
| | | Symptoms of diabetes and |
| | | casual plasma glucose |
| | | concentration ≧200 mg/dl |

^{‡}Midrange values indicating impaired glucose tolerance (IGT), or impaired fasting glucose (IFG).
*A diagnosis of diabetes must be confirmed, on a subsequent day, by measurement of FPG, 2-h PG, or random plasma glucose (if symptoms are present). Fasting is defined as no caloric intake for at least 8 h.
^{†}This test requires the use of a glucose load containing the equivalent of 75 g anhydrous glucose dissolved in water. 2-h PG, 2-h postload glucose.

An insulin resistant subject is a subject, e.g., a human subject, who has been diagnosed as being insulin resistant (or would be diagnosed as being insulin resistant) by a skilled medical practitioner or researcher. An insulin resistant subject can be identified, for example, by determining fasting glucose and/or insulin levels in said subject. In a preferred embodiment, an insulin resistant subject has a fasting glucose level of less than 110 mg/dL and has a fasting insulin level of greater that 30 mU/L.

The effectiveness of treatment of a subject with an insulin response modulator can be monitored by (i) detecting the level of insulin responsiveness or, alternatively, glucose tolerance in the subject prior to treating with an appropriate modulator; (ii) detecting the level of insulin responsiveness or, alternatively, glucose tolerance in the subject prior post treatment with the modulator; (iii) comparing the levels pre-administration and post administration; and (iv) altering the administration of the modulator to the subject accordingly. For example, increased administration of the modulator may be desirable if the subject continues to demonstrate insensitive insulin responsiveness.

Pharmaceutical Preparations

Modulators of the present invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the 'site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal, models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

Pharmaceutical preparations of gene therapy vectors can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

The following examples are intended to illustrate but not limit the invention. While they are typical of those that might be used, other procedures known to those skilled in the art may alternatively be used. Other uses for the methods of the invention will be apparent to one of ordinary skill in the art from the following Examples and Claims.

### EXEMPLIFICATION

### Example 1

### Analysis of FHOS mRNA Expression in Adipocytes from Subjects having Diabetes or Insulin Resistance

To evaluate the relative expression level of the FHOS mRNA among human subjects of different phenotypes, relative quantitative RT-PCR was performed using RNA from human adipocytes and primers specific for the human FHOS gene sequence. This system compares the amount of RT-PCR product, which reflects the level of mRNA expression, among individual samples and normalizes the product to the amount of product generated from amplification of the 18S rRNA, which should be constant among samples. Human subjects of different phenotypes where analyzed. Preadipocytes from lean subjects (BMI<25), obese insulin resistant subjects (BMI>30, fasting glucose<100 mg/dL, fasting insulin>30 mU/L), and obese diabetic subjects (BMI>30, diagnosed with type II diabetes) were grown and expanded in culture to the same passage number and then differentiated to adipocytes. Total RNA was then isolated from the adipocytes and used as templates in the relative quantitative RT-PCR using amplification of the 18S rRNA as an internal control. For the reverse transcription reaction random decamers were used, and for the PCR amplification step FHOS-specific primers were used. The PCR cycling conditions were optimized for the FHOS template.

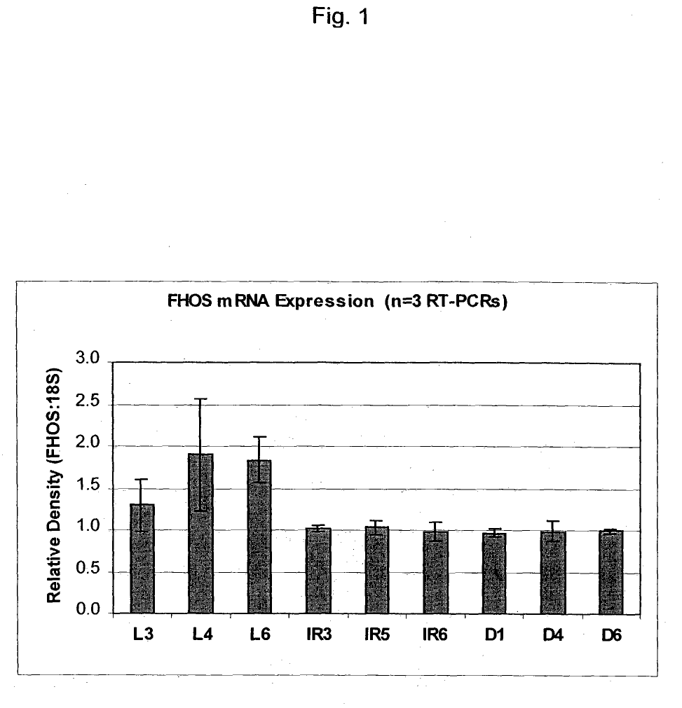

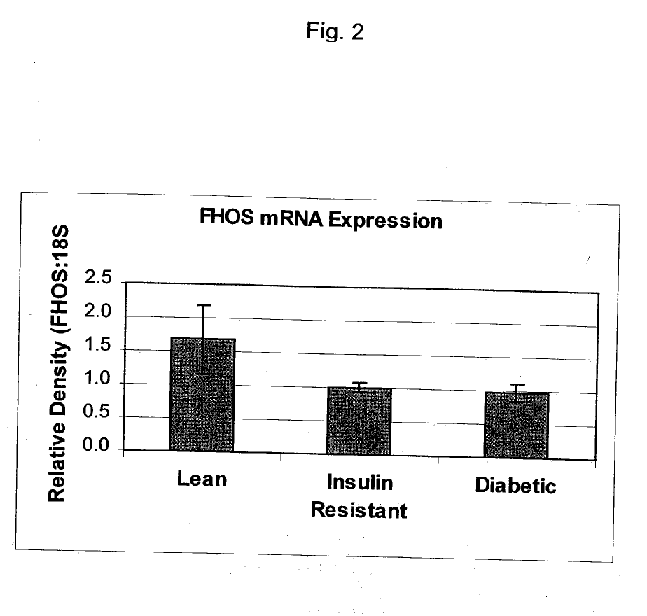

Using this system, three independent RT-PCRs were performed and reproducible results were obtained (FIG. 1). Expression of the FHOS mRNA was reduced in both insulin resistant and diabetic individuals compared to lean subjects. Expression of the FHOS mRNA in adipocytes from obese insulin resistant and diabetic subjects was similar. Data are averaged and presented in FIG. 2.

### Example 2

### Analysis of FHOS Protein Expression in Adipocytes from Subjects having Diabetes or Insulin Resistance

To analyze expression of the FHOS protein among these phenotypic groups, immunoblotting of equivalent amounts of cellular protein was performed. An antibody raised against a synthetic peptide corresponding to amino acids 1152-1166 of FHOS was affinity purified and used for these purposes. Preliminary data demonstrate that FHOS protein levels are decreased in lysates from insulin-resistant subjects.

### Example 3

### Effect of FHOS in Diabetes Disease Models

The foregoing Examples demonstrate that FHOS expression in decreased or downregulated in subjects having various insulin-related disorders. These data suggest that increasing FHOS expression or activity could have important applications for the treatment of, for example, type II diabetes, insulin resistance, obesity and disorders related to obesity. To pursue these potential applications, the following approaches can be taken.

Demonstration of the Efficacy of FHOS Activators in Rodent Models of Obesity/Diabetes

Rodents, e.g., mice or rats, serving as models for diabetes can be treated with FHOS activators to determine whether activation of FHOS reduces or ameliorates the symptoms of either diabetes (or insulin resistance) in these animals.

Rodents with diabetes are treated with one or more FHOS activators in a therapeutically effective dose. FHOS levels in treated and control rodents can be assessed by Western blot analysis using antibodies specific for FHOS. Levels of molecules characteristic for diabetes or insulin resistance, such as glucose, insulin, lipids, and creatine kinase can be assessed in serum samples taken from treated and control animals. Functional assays for diabetes including, but not limited to, an insulin sensitivity assay, a glucose tolerance assay and an ex-vivo glucose uptake by isolated muscle cell assay can be performed to monitor the effect of the activator on treated and non-treated animals. Moreover, body weight, and/or fat pad weights can be assayed in these animals. Similarly, muscle and fat cell differentiation can be observed in these animals. Analysis of such studies should enable a determination of the overall effect of the activation of FHOS on the progression of diabetes in animal models of the disease.

All patents, published patent applications and other published references disclosed herein are hereby expressly incorporated herein in their entireties by reference.

Equivalents

Those skilled in the art will recognize, or will be able to ascertain using no more than routine experimentation, many equivalents to the specific embodiments of the invention described herein. Such equivalents are intended to be encompassed by the following claims.

We claim:

1. A method of increasing expression of FHOS in a subject comprising administering to the subject a FHOS activator, such that FHOS expression is increased.

2. The method of claim 1, wherein FHOS mRNA levels are increased

3. The method of claim 1, wherein FHOS protein levels are increased.

4. A method of treating diabetes in a subject comprising administering to the subject a FHOS activator.

5. The method of claim 4, wherein the subject is suffering from type II diabetes.

6. A method of treating insulin resistance in a subject comprising administering to the subject a FHOS activator.

7. The method of any one of claims 1-6, wherein the FHOS activator is selected from the group consisting of a FHOS nucleic acid molecule, a plasmid comprising a FHOS nucleic acid molecule, a FHOS adenovirus and a FHOS retrovirus.

8. The method of any one of claims 1-6, wherein the FHOS activator is selected from the group consisting of a FHOS protein or biologically active portion thereof, an antibody or biologically active portion thereof, a peptide, a peptidimetic, a non-peptide oligomer and a small molecule.

9. A method for identifying a compound suitable for use in treating diabetes or insulin resistance in a subject, said method comprising contacting a cell capable of expressing FHOS mRNA with a test compound and determining the effect of the test compound on expression of FHOS mRNA, wherein a stimulatory effect is indicative of the compound being suitable for use in treating diabetes or insulin resistance in said subject.

10. A method for identifying a compound suitable for use in treating diabetes or insulin resistance in a subject, said method comprising contacting a cell capable of expressing FHOS protein with a test compound and determining the effect of the test compound on expression of FHOS protein, wherein a stimulatory effect is indicative of the compound being suitable for use in treating diabetes or insulin resistance in said subject.

11. A method for identifying a compound suitable for use in treating diabetes or insulin resistance in a subject, said method comprising contacting a cell which expresses FHOS protein with a test compound and determining the effect of the test compound on a biological activity of the FHOS protein, wherein a stimulatory effect is indicative of the compound being suitable for use in treating diabetes or insulin resistance in said subject.

12. A method for identifying a compound suitable for use in treating diabetes or insulin resistance in a subject, said method comprising contacting a FHOS protein or biologically active portion thereof with a test compound and determining the effect of the test compound on a biological activity of the FHOS protein or portion, wherein a stimulatory effect is indicative of the compound being suitable for use in treating diabetes or insulin resistance in said subject.

13. A compound identified by the method of any one of claims 9-12.

14. The compound of claim 13 formulated with a pharmaceutically-acceptable carrier.

15. A method of treating diabetes in a subject comprising administering to the subject compound identified by the method of any one of claims 9-12.

16. The method of claim 15, wherein the subject is suffering from type II diabetes.

17. A method of treating insulin resistance in a subject comprising administering to the subject a compound identified by the method of any one of claims 9-12.

18. A method of increasing FHOS expression or activity in a cell comprising contacting said cell with a FHOS activator.

19. A method of increasing FHOS expression or activity in a cell comprising contacting said cell with a compound identified by the method of any one of claims 9-12.

20. A pharmaceutical composition comprising a cell which overexpreses FHOS protein and a pharmaceutically-acceptable carrier.

21. The method of any one of claims 18-20, wherein the cell is a muscle cell or a precursor thereof.

22. The method of any one of claims 18-20, wherein the cell is an adipocyte or a precursor thereof.

23. A method of treating a subject having diabetes or an insulin-resistant subject comprising obtaining cells from said subject, treating said cells with an FHOS activator, and administering said treated cells to said subject such that diabetes or insulin-resistance in said subject is treated.

24. The method of claim 23, wherein the FHOS activator is selected from the group consisting of a FHOS nucleic acid molecule, a plasmid comprising a FHOS nucleic acid molecule, a FHOS adenovirus, and a FHOS retrovirus.


SEQUENCE LISTING (2 sequences)

SEQ ID NO: 1 - DNA, 3775 bp, Homo sapiens
tgagccggccgcagagccatggcgggcggggaagaccgcggggacggagagccggtatcagtggtgaccgtgagggtgcagtacctggaagacaccgaccccttcgcatgtgccaactttccggagccgcgccgggcccccacctgcagcctggacggggcgctgcccttgggcgcgcagatacccgcggtgcaccgcctgctgggagcgccgctcaagttggaggattgtgctctgcaagtgtctccctccggatactacctggacaccgagctgtccctggaagagcagcgggagatgctggagggcttctatgaagagatcagcaaagggcggaagcccacgctgatccttcggacccagctctctgtgagggtcaacgctatcttggaaaagctgtatagctccagtggtcctgagctccgccgctccctcttctcactgaagcagatcttccaggaggacaaagacctggtgcctgaatttgtgcattcagaggggctgagctgcctgatccgtgtgggtgctgctgccgaccacaactaccagagctacatccttagagcgctcggccagctgatgctctttgtggatggaatgctgggggtggtggcccacagtgacactattcagtggctgtacacattgtgtgccagcctgtcccgcttggtggtgaagacagccctgaagctgctgttggtgtttgtagaatactccgaaaacaacgcaccgctgttcatccgtgcagtgaactctgtggccacgaccaccggtgctcctccctgggccaatctggtgtccatcctggaggagaagaatggcgctgaccctgagttgttggtgtacacggtcaccctcatcaacaagacgctggcggcgctcccggaccaggactccttctacgatgtgacggatgcactggagcagcagggcatggacacgctggtccagcgccacctgggcactgcgggcactgacgtcgacctgcgcacgcagcttgtgctctacgagaacgccctgaaattggaggatggagacatcgaagaagccccaggcgctggtgggcggcgggaacgacgaaagccttcttctgaggagggcaagaggagccgccgttctctggaaggcgggggctgccccgcgcgtgccccggaacctggccccacaggccccgcctcaccggtaggccccacctcttccaccggccccgccctgctgacaggccccgcctccagccctgtgggccctccctccggtctccaagcttcagtgaacctttttcctaccatctctgtggcaccctcagctgacacctccagcgagaggagcatctacaaagcccggttcctggagaatgtggcggcagcagaaacagagaagcaggttgcgctggcccagggccgggcagagacacttgccggggccatgcccaatgaggcgggtggacacccagatgcccggcaactctgggactccccagagacagcccctgcagccagaacaccccagagccctgccccctgtgtcctgctccgggcccagcgaagccttgcaccagagcccaaggagccactgataccagcaagccccaaggctgagcccatctgggagctccctacccgtgcacccaggctctctattggggacctggacttttcagatctaggggaggatgaagaccaggacatgctgaatgtagagtctgtggaggctgggaaagacatcccagctccctcacccccactgcccctgctctcgggagtacccccccctcccccacttccacctcccccacccatcaaaggccccttcccaccacctccacctctacctctggctgcccctcttccccattcagtgcctgacagctcagccctccccactaagaggaagacagtaaaacttttctggcgtgacgtgaagctggctgggggccatggagtctctgcaagccgctttgggccctgcgccaccctctgggcttcactggaccctgtctcagtggacacggcccgactggaacacctctttgagtctcgtgccaaagaggtgctgccctccaagaaagctggagagggccgccggacaatgaccacagtgctggaccccaagcgcacgaacgccatcaacatcggcctaaccacactgccacctgtgcatgtcattaaggctgctctgctcaactttgatgagtttgctgtcagcaaggatggcattgagaagctactgaccatgatgcccacggaggaagagcggcagaagattgagggagcccagctggccaaccctgacatacccctgggcccagccgagaacttcctgatgactcttgcctccattggcggcctcgctgctcgtctacaactctgggccttcaagctggactatgacagcatggagcgggaaattgctgagccactgtttgacctgaaagtgggtatggaacagctggtacagaatgccaccttccgctgcatcctggctaccctcctagctgtgggcaacttcctcaatggctcccagagcagcggctttgagctgagctacctggagaaggtgtcagatgtgaaggacacggtgcgtcgacagtcactgctacaccatctctgctccctagtgctccagacccggcctgagtcctctgacctctattcagaaatccctgccctgacccgctgtgccaaggtggactttgaacagctgactgagaacctggggcagctggagcgccggagccgggcagccgaggaaagcctgcggagcttggccaagcatgagctggccccagccctgcgtgcccgcctcacccacttcctggaccagtgtgcccgccgtgttgccatgctaaggatagtgcaccgccgtgtctgcaataggttccatgccttcctgctctacctgggctacaccccgcaggcggcccgtgaagtgcgcatcatgcagttctgccacacgctgcgggaatttgcgcttgagtatcggacttgccgggaacgagtgctacagcagcagcagaagcaggccacataccgtgagcgcaacaagacccggggacgcatgatcaccgagacagagaagttctcaggtgtggctggggaagcccccagcaacccctctgtcccagtagcagtgagcagcgggccaggccggggagatgctgacagtcatgctagtatgaagagtctgctgaccagcaggcttgaggacaccacacacaatcgccgcagcagaggcatggtccagagcagctccccaatcatgcccacagtggggccctccactgcatccccagaagaacccccaggctccagtttacccagtgatacatcagatgagatcatggaccttctggtgcagtcagtgaccaagagcagtcctcgtgccttagctgctagggaacgcaagcgttcccgcggcaaccgcaagtctttgagaaggacgttgaagagtgggctcggagatgacctggtgcaggcactgggactaagcaagggtcctggcctggaggtgtgaaggtgctgtatcccggaaatctatctggaccctggactgcagtgcaggagatgacagagtgaggagggcccagagcagaattctggccccagaactctgtgcccaggagccatgccttgagcagtattagccgtgtgtgtatgcatgtgagtgtgtgtgtatgtgtgtgtgtgcatgcatatgcatgtgcatgtgtgtgagctccttgaacgcacggagcaaaataaaattttcttagctaatcccaaaaaaaaaaaaaaaa

SEQ ID NO: 2 - protein, 1164 aa, Homo sapiens
MAGGEDRGDGEPVSVVTVRVQYLEDTDPFACANFPEPRRAPTCSLDGALPLGAQIPAVHRLLGAPLKLEDCALQVSPSGYYLDTELSLEEQREMLEGFYEEISKGRKPTLILRTQLSVRVNAILEKLYSSSGPELRRSLFSLKQIFQEDKDLVPEFVHSEGLSCLIRVGAAADHNYQSYILRALGQLMLFVDGMLGVVAHSDTIQWLYTLCASLSRLVVKTALKLLLVFVEYSENNAPLFIRAVNSVATTTGAPPWANLVSILEEKNGADPELLVYTVTLINKTLAALPDQDSFYDVTDALEQQGMDTLVQRHLGTAGTDVDLRTQLVLYENALKLEDGDIEEAPGAGGRRERRKPSSEEGKRSRRSLEGGGCPARAPEPGPTGPASPVGPTSSTGPALLTGPASSPVGPPSGLQASVNLFPTISVAPSADTSSERSIYKARFLENVAAAETEKQVALAQGRAETLAGAMPNEAGGHPDARQLWDSPETAPAARTPQSPAPCVLLRAQRSLAPEPKEPLIPASPKAEPIWELPTRAPRLSIGDLDFSDLGEDEDQDMLNVESVEAGKDIPAPSPPLPLLSGVPPPPPLPPPPPIKGPFPPPPPLPLAAPLPHSVPDSSALPTKRKTVKLFWRDVKLAGGHGVSASRFGPCATLWASLDPVSVDTARLEHLFESRAKEVLPSKKAGEGRRTMTTVLDPKRTNAINIGLTTLPPVHVIKAALLNFDEFAVSKDGIEKLLTMMPTEEERQKIEGAQLANPDIPLGPAENFLMTLASIGGLAARLQLWAFKLDYDSMEREIAEPLFDLKVGMEQLVQNATFRCILATLLAVGNFLNGSQSSGFELSYLEKVSDVKDTVRRQSLLHHLCSLVLQTRPESSDLYSEIPALTRCAKVDFEQLTENLGQLERRSRAAEESLRSLAKHELAPALRARLTHFLDQCARRVAMLRIVHRRVCNRFHAFLLYLGYTPQAAREVRIMQFCHTLREFALEYRTCRERVLQQQQKQATYRERNKTRGRMITETEKFSGVAGEAPSNPSVPVAVSSGPGRGDADSHASMKSLLTSRLEDTTHNRRSRGMVQSSSPIMPTVGPSTASPEEPPGSSLPSDTSDEIMDLLVQSVTKSSPRALAARERKRSRGNRKSLRRTLKSGLGDDLVQALGLSKGPGLEV